(19) United States
(12) Patent Application Publication
(10) Pub. No.: US 2015/0014397 A1
(43) Pub. Date: Jan. 15, 2015

(54) METHOD FOR BONDING A TANTALUM STRUCTURE TO A COBALT-ALLOY SUBSTRATE

(71) Applicant: Zimmer, Inc., Warsaw, IN (US)

(72) Inventors: Gregory M. Hippensteel, South Whitley, IN (US); Lawrence F. Peek, Warsaw, IN (US); Jeffrey P. Anderson, Warsaw, IN (US); Devendra Gorhe, Warsaw, IN (US); Steve M. Allen, Winona Lake, IN (US); Clarence M. Panchison, Warsaw, IN (US); David M. Miller, Warsaw, IN (US); Joel G. Scrafton, Syracuse, IN (US); Casey Harmon, Warsaw, IN (US)

(21) Appl. No.: 14/500,139

(22) Filed: Sep. 29, 2014

Related U.S. Application Data
(63) Continuation of application No. 13/092,169, filed on Apr. 22, 2011.
Application No. 13/092,169 is a continuation-in-part of application No. 11/870,205, filed on Oct. 10, 2007, now Pat. No. 8,608,049.

(51) Int. Cl. B23K 20/02; B23K 20/24

(52) U.S. Cl. CPC B23K 20/023; B23K 20/24; B23K 2203/18; A61F 2310/00544; A61F 2002/30929
USPC 228/173.1

(57) ABSTRACT

Methods for bonding a porous tantalum structure to a substrate are provided. The method includes placing a compressible or porous interlayer between a porous tantalum structure and a cobalt or cobalt-chromium substrate to form an assembly. The interlayer comprising a metal or metal alloy that has solid state solubility with both the substrate and the porous tantalum structure. Heat and pressure are applied to the assembly to achieve solid state diffusion between the substrate and the interlayer and the between the porous tantalum structure and the interlayer.

The present application is a continuation-in-part of U.S. patent application Ser. No. 11/870,205, filed Oct. 10, 2007, which is hereby incorporated herein by reference.

### FIELD OF THE INVENTION

This invention relates generally to orthopedic implants, and more particularly relates to a method for bonding a porous tantalum structure to cobalt or a cobalt-alloy orthopedic implant.

### BACKGROUND OF THE INVENTION

Orthopedic implants are often utilized to help their recipients recover from injury or disease. To promote quick recovery, orthopedic implants are designed to cooperate with the body's natural inclination to heal itself. Some orthopedic implants are designed to foster osseointegration. As is known in the art, osseointegration is the integration of living bone within a man-made material, usually a porous structure. Cells in the recipient form new bone within the pores of the porous structure. Thus, the porous structure and the bone tissue become intermingled as the bone grows into the pores. Accordingly, orthopedic implants may include a porous surface to enhance fixation between the orthopedic implant and adjacent tissue. Of course, the faster the surrounding tissue grows into the porous surface, the sooner the patient may begin to resume normal activities. However, the manufacture of the orthopedic implants with porous structures is not without difficulty.

Orthopedic implants are usually made from various metals. One difficulty encountered during manufacturing is bonding separate components, each made of a different metal, together. For example, cobalt is a popular metal used to make orthopedic implants, and other popular metals include alloys of cobalt with other metals, such as chromium. The porous structure may be made from an entirely different metal, such as tantalum. In this case, bonding the porous metal to the orthopedic implant involves bonding tantalum to cobalt or to cobalt-chromium alloys. Bonding these two metals together has proved to be particularly problematic.

Thus, there is a need for an improved method of bonding of porous structures, specifically tantalum, to cobalt and cobalt-alloy implants such that the bond has sufficient strength while the corrosion resistance of the metals in the resulting implant are maintained.

### SUMMARY OF THE INVENTION

The present invention provides a method for bonding a porous tantalum structure to a substrate. In one embodiment, the method comprises providing (i) a substrate comprising cobalt or a cobalt-chromium alloy; (ii) interlayer consisting essentially of at least one of hafnium, manganese, niobium, palladium, zirconium, titanium, or alloys or combinations thereof; and a porous tantalum structure, and applying heat and pressure for a time sufficient to achieve solid-state diffusion between the substrate and the interlayer and solid-state diffusion between the interlayer and the porous tantalum structure.

In one aspect, the disclosure provides a method for bonding a porous tantalum structure to a substrate. The method comprises positioning a compressible interlayer between a porous tantalum structure and a substrate comprising cobalt or cobalt-chromium to form an assembly wherein the compressible interlayer consists essentially of a metal or alloy that exhibits solid solubility with the porous tantalum structure and the substrate. Heat and pressure are applied to the assembly for a time sufficient to achieve solid-state diffusion between the substrate and the compressible interlayer and solid state diffusion between the compressible interlayer and the porous tantalum structure.

In another aspect, a method for bonding a porous tantalum structure to a substrate is provided. The method includes providing a porous tantalum structure in a first configuration and providing a substrate comprising cobalt or cobalt-chromium. A porous interlayer is applied to a surface of the porous tantalum structure to form a subassembly wherein the porous interlayer comprises a metal or alloy that is soluble in the solid state with both the porous tantalum structure and the substrate. The subassembly is bent into a second configuration and a surface of the substrate is brought into contact with the interlayer to create an assembly. Heat and pressure are applied to the assembly for a time sufficient to achieve solid-state diffusion between the substrate and the interlayer and solid state diffusion between the interlayer and the porous tantalum structure.

In yet another aspect, the present disclosure provides an assembly for forming a medical implant. The assembly comprises a porous tantalum structure and a substrate comprising cobalt or cobalt-chromium alloy. The assembly also includes a compressible interlayer positioned between the porous tantalum structure and the substrate, wherein the compressible interlayer consists essentially of a metal or alloy that exhibits solid solubility with the porous tantalum structure and the substrate.

In a further aspect, the present disclosure provides a medical implant comprising a porous tantalum structure and a substrate made of cobalt or cobalt-chromium alloy. The implant further includes a compressed interlayer between a surface of the porous tantalum structure and a surface of the substrate. The compressed interlayer consists essentially of a metal or alloy that exhibits solid solubility with the porous tantalum structure and the substrate.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 depicts a cross-sectional view of one embodiment of an assembly comprising a porous tantalum structure, a pre-formed sheet interlayer, and a substrate;

FIG. 2 depicts a cross-sectional view of another embodiment of an assembly comprising a porous tantalum structure, a coating interlayer, and a substrate;

FIGS. 3 and 4 are photomicrographs corresponding to the embodiments of FIGS. 1 and 2, respectively, following heating and pressing the assembly to bond the porous tantalum structure to the interlayer and the interlayer to the substrate;

FIG. 5 is a perspective view of an exemplary embodiment of a cobalt-chromium femoral implant that may have a porous tantalum structure bond thereto in accordance with the methods of the present disclosure;

FIG. 6 is an exploded perspective view of one embodiment of a femoral implant construct of the present disclosure including a porous tantalum structure and a substrate;

FIG. 7 is a planar view of the porous tantalum structure of FIG. 6 shown in a substantially flat configuration;

FIG. 8 is perspective view of the femoral implant construct of FIG. 6;

FIG. 9 is a photomicrograph showing a porous tantalum structure having a coated interlayer applied thereto;

FIG. 10 is a photomicrograph showing a compressed interlayer bonded to a tantalum structure and to a substrate;

FIG. 11 is a laser holography image of a construct made using a solid interlayer; and

FIG. 12 is a laser holography image of a construct made using a compressible interlayer.

### DETAILED DESCRIPTION

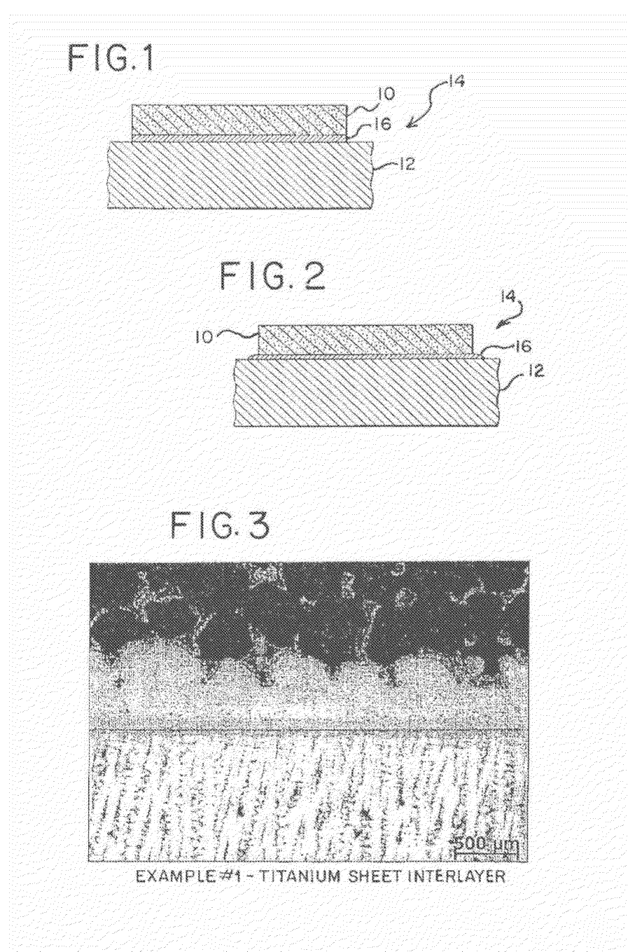

In accordance with the present invention and with reference to FIGS. 1 and 2, a method for bonding a porous tantalum structure 10 to a substrate 12 generally begins by constructing an assembly 14 comprising an interlayer 16 placed on the surface of the substrate 12 and the porous tantalum structure 10 placed onto the interlayer 16. It will be appreciated that the assembly 14 may be constructed by placing the individual components 10, 12, 16 together in any order that results in the interlayer 16 positioned between and in contact with the substrate 12, and the porous tantalum structure 10, as shown in FIGS. 1 and 2. In other words, the placement order is not limited to those orders described herein.

The porous tantalum structure 10 may be TRABECULAR METAL®, available from Zimmer Inc., Warsaw, Ind. The porous tantalum structure 10 is configured to facilitate osseointegration. The porous tantalum structure 10 may have a pore size, pore continuity, and other features for facilitating bone tissue growth into the pores, as is known in the art.

The substrate 12 may be a cast or a wrought cobalt or cobalt chromium alloy fabricated in a shape according to the requirements for the specific orthopedic application. For example, the substrate 12 may be cast of cobalt in the shape of a total hip replacement implant. Other implants may include implants for the ankle, elbow, shoulder, knee, wrist, finger, and toe joints or other portions of the body that may benefit from a substrate 12 having a porous tantalum structure 10 bonded thereto.

With no intent to be bound by theory, tantalum and cobalt metals are not readily soluble, that is, the documented solid solubility of tantalum into cobalt is insufficient to form the necessary bond strength demanded by applications within the human body. In fact, certain stoichiometries of tantalum with cobalt may prevent solid-state diffusion of tantalum into cobalt and vice versa. Therefore, in accordance with the method of the present disclosure, the interlayer 16 comprises a metal that ready forms solid solutions with both tantalum and cobalt or cobalt-chromium alloys. For example, the interlayer 16 may be any one or an alloy of metals, such as, hafnium, manganese, niobium, palladium, zirconium, titanium, or other metals or alloys that exhibit solid solubility with tantalum at temperatures less than the melting temperature of the substrate 12, the interlayer 16, or the porous tantalum structure 10.

The assembly 14, as shown FIGS. 1 and 2, may be put together by applying the interlayer 16 to the substrate 12. One skilled in the art will observe that the interlayer 16 may require pre-shaping to improve the contact area between the surface of the substrate 12 and the surface of interlayer 16 prior to applying the interlayer 16 to the substrate 12. Alternatively, the interlayer 16 may be press formed onto the substrate 12 such that the interlayer 16 conforms to the surface of the substrate 12. The surfaces of all components 10, 12, 16 may be cleaned prior to assembly 14 to reduce corrosion and improve solid-state diffusion bonding.

With continued reference to FIGS. 1 and 2, following application of the interlayer 16 to the substrate 12, the porous tantalum structure 10 may be placed on the interlayer 16 thus forming the assembly 14. Similar to pre-shaping the interlayer 16 to conform to the substrate 12, the porous tantalum structure 10 may be formed in a shape to maximize surface-to-surface contact to facilitate solid-state diffusion with the interlayer 16.

Heat and pressure are applied to the assembly 14 sufficient for solid-state diffusion to take place between the substrate 12 and the interlayer 16 and between the interlayer 16 and the porous tantalum structure 10. As is known to those skilled in the art, solid-state diffusion is the movement and transport of atoms in solid phases. Solid-state diffusion bonding forms a monolithic joint through formation of bonds at an atomic level due to transport of atoms between two or more metal surfaces. Heat and pressure may be supplied to the assembly 14 with a variety of methods known in the art. For example, the assembly 14 may be heated electrically, radiantly, optically, by induction, by combustion, by microwave, or other means known in the art. Pressure may be applied mechanically by clamping the assembly 14 together prior to insertion of the assembly 14 into a furnace, or pressure may be applied via a hot pressing system capable of applying pressure once the assembly 14 reaches a target temperature, as is known in the art. Furthermore, hot pressing may include hot isostatic pressing, also known in the art.

Referring now to FIG. 1, in one embodiment, the interlayer 16 is a pre-formed sheet of commercially pure titanium at least a bout 0.016 inches (about 0.04064 centimeter) thick. In another embodiment, the pre-formed sheet of commercially pure titanium is at least about 0.020 inches (about 0.0508 centimeter) thick for improved bond strength. It will be observed that the interlayer 16 may be positioned directly beneath the porous tantalum structure 10. In other words, it is not necessary to cover the entire substrate 12 with the interlayer 16 to bond the porous tantalum structure 10 at a single location. Furthermore, it will also be observed that the corrosion resistance and the strength of the substrate 12 are not negatively impacted if the porous tantalum structure 10 touches those areas not covered by the interlayer 16 during heating. Thus, the porous tantalum structure 10 may be bonded to multiple separate areas on the surface of the substrate 12 with multiple separate areas of interlayer 16. One skilled in the art will appreciate that the position of the porous tantalum structure 10 may be dictated by the patients physiological requirements.

In one embodiment, the assembly 14 is clamped together by applying a pressure of at least approximately 200 pounds per square inch (psi) (approximately 1.38 MPa). However, pressures greater than approximately 200 psi may be applied up to the compressive yield strength of the any of the substrate 12, the interlayer 16, or the porous tantalum structure 10. Ordinarily, the porous tantalum structure 10 has the lowest compressive yield strength, for example, 5,800 psi for TRABECULAR METAL®.

The clamped assembly 14 is then heated to at least about 540° C. (about 1004 degree Fahrenheit) in vacuum or in another sub-atmospheric pressure of an inert atmosphere, in any case, the clamped assembly 14 is heated to less than the melting temperature of any of the components 10, 12, 16 and, in most cases, is at least about 800° C. (about 1472 degree Fahrenheit) but less than about 1000° C. (about 1832 degree Fahrenheit) in vacuum. One skilled in the art will observe that the higher the temperature, the less time it will take to achieve solid-state diffusion bonding. The time required to achieve solid-state diffusion bonding may be as little as less than 1 hour to as long as 48 hours and will depend on the metals involved, the temperatures, atmosphere, and the pressures applied.

One heated to temperature, and after a time sufficient to achieve solid-state diffusion between the porous tantalum structure 10 and the interlayer 16 and between the interlayer 16 and the substrate 12, a construct is formed. The construct may comprise the substrate 12 bonded to the interlayer 16 and the interlayer 16 bonded to the porous tantalum structure 10. FIG. 3 is a photomicrograph of a portion of the construct formed according to one embodiment of the method, described above, with a porous tantalum structure 10 (top) bonded to a titanium sheet interlayer 16 (middle) bonded to a cobalt-chromium substrate 12 (bottom).

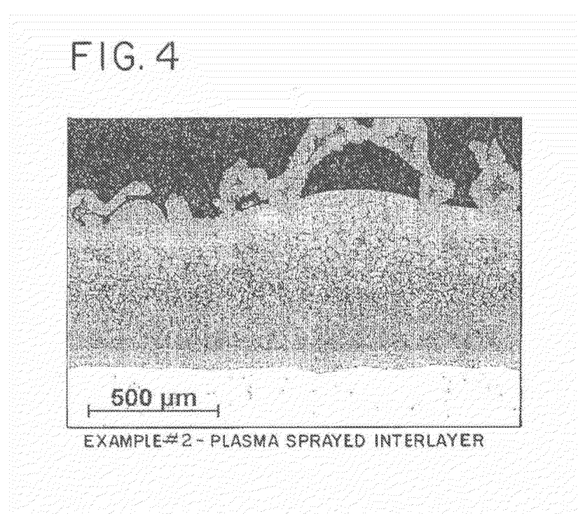

With reference now to FIG. 2, in another embodiment, the interlayer 16 is a coating applied to the surface by, for example, thermal spray, plasma spray, electron beam deposition, laser deposition, cold spray, or other method of forming the coatings on a substrate 12. In one exemplary embodiment, the coating interlayer 16 is applied via vacuum plasma spraying, as is known in the art. The substrate 12 may be masked and then grit blasted to prepare the surface of the substrate 12 for vacuum plasma spraying. In one exemplary embodiment, the substrate 12 is masked and then grit blasted with alumina (aluminum oxide) grit for increased corrosion resistance of the construct subsequent to bonding with the interlayer 16. In another exemplary embodiment, the coating interlayer 16 comprises titanium sprayed to a thickness of at least about 0.010 inches (about 0.0254 centimeter) thick. In another embodiment, for increased bond strength, the titanium coating interlayer 16 is at least about 0.020 inches (about 0.0508 centimeter) thick. In the vacuum plasma sprayed embodiments, a porosity level is between about 20% and about 40% for ease of vacuum plasma spray processing while maintaining sufficient corrosion resistance. In other embodiments, the porosity may be at least about 5%. In still other embodiments, the porosity may be at least about 20%, at least about 30% or at least about 40%. In another embodiment, the porosity may be between about 30% and about 40%. A plasma sprayed interlayer typically includes adjoining metal particles or ligand defining pores therebetween. As explained in more detail below, the porosity of the interlayer allows for compressibility of the interlayer, which compressibility may be advantageous and desired in some applications. FIG. 4 is a photomicrograph of a portion of a construct formed according to one embodiment of the method described above, showing a portion of a construct comprising a porous tantalum structure 10 (top) bonded to a titanium vacuum plasma sprayed interlayer 16 (middle) bonded to a cobalt-chromium substrate 12 (bottom).

Coated interlayer 16 may be coated on either the porous tantalum structure 10 or the substrate 12 by any of the coating processes disclosed above and, in one embodiment, coated interlayer 16 is applied by plasma spraying. When the surface of substrate 12 is geometrically complex, it may be difficult to form a coated interlayer of uniform thickness on the surface of the substrate. A coated interlayer of non-uniform thickness may result in undesired incongruency between the surfaces of the substrate and tantalum porous structure. It also may result in incomplete bonding of the tantalum porous structure to the substrate and undesired surface deviations.

As used herein a “geometrically complex” surface of a substrate is a surface that is other than a simple continuous flat surface. Such geometrically complex surfaces may include, but are not limited to, surfaces that include two or more flat sections that project at an angle with respect to each other, surfaces that include multiple flat sections wherein the flat sections project at angles with respect to adjacent sections, non-flat surfaces, rounded surfaces, concave surfaces, convex surfaces, and combinations thereof. When it is difficult to coat the interlayer on the surface of the substrate because of the surface's geometry, or for some other reason, the interlayer may be coated onto a surface of the porous tantalum structure instead of a surface of the substrate.

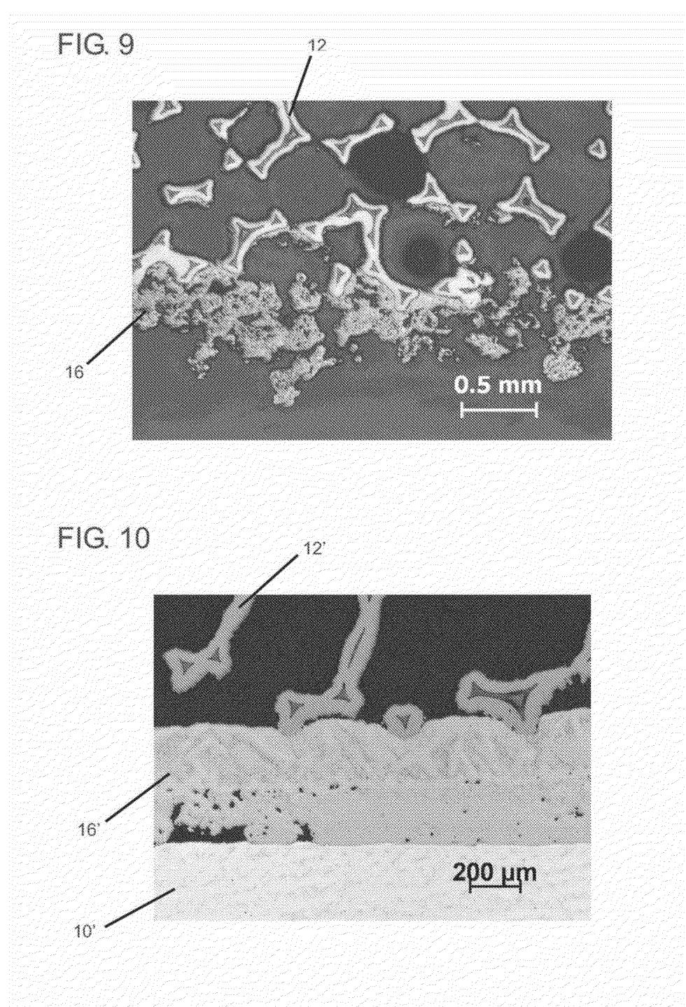

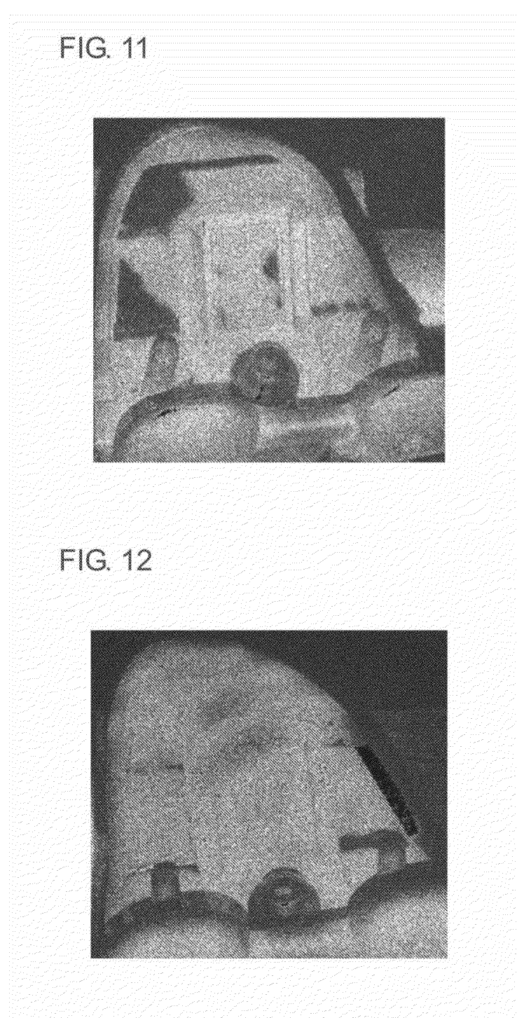

One concern with applying a coated interlayer 16 to a surface of the porous tantalum structure 12 is that the potential for coated interlayer 16 to occlude or block the pores of porous tantalum structure 12. For example, during the plasma spraying process, the metal which forms interlayer 16 is formed into liquid particles, which particles are applied to porous tantalum structure 12. It was thought that such liquid particles would enter the pores of porous tantalum structure 12 where the particles would solidify and occluded the pores of tantalum structure 12. However, in accordance with the methods is closed herein, coated interlayer 16 can be applied or coated onto porous tantalum structure 12 without causing significant pore occlusion. FIG. 9 is a microphotograph of a portion of a tantalum structure 12 having a plasma sprayed interlayer 16 coated thereon. As shown in FIG. 9, the interlayer 16 does not significantly occlude the porous tantalum structure 12.

A construct comprising a porous tantalum structure 10 of TRABECULAR METAL® bonded to a titanium interlayer 16 bonded to a cobalt-chromium substrate 12 was characterized by tensile strength testing. Nearly all failure separations occurred in the porous tantalum structure 10. Tensile stresses measured at separation on constructs formed according to the previously described embodiments were routinely above 2900 psi.

One skilled in the art will observe that heating and applying pressure include multiple heating and pressurizing processes. For example, the porous tantalum structure 10 may be assembled with the interlayer 16 and bonded thereto, according to one embodiment of the method, to form a subassembly. That subassembly may then be bonded to the substrate 12 according to another embodiment of the method. The reverse procedure may also be used. That the interlayer 16 may be bonded to the substrate 12 to form a subassembly with subsequent bonding of the porous tantalum structure 10 to the interlayer portion of the subassembly. Therefore, embodiments of the method may account for different diffusion coefficients between the components 10, 12, 16 which may allow for more consistent, higher strength bonds between the substrate 12 and interlayer 16 and between the interlayer 16 and the porous tantalum structure 10. By way of further example and not limitation, diffusion bonding of a titanium interlayer 16 to a cobalt-chromium substrate 12 at an elevated temperature and pressure may take longer than diffusion bonding of the titanium interlayer 16 to a porous tantalum structure 10 at similar pressures and temperatures. Thus, by diffusion bonding the titanium interlayer 16 to the cobalt-chromium substrate 12 to form a subassembly and then diffusion bonding the porous tantalum structure 10 to the subassembly, a diffusion bond depth between the titanium interlayer 16 and the cobalt-chromium substrate 12 may be substantially the same as a diffusion bond depth between the titanium interlayer 16 and the porous tantalum structure 10. In contrast, if the porous tantalum structure 10, the titanium interlayer 16, and the cobalt-chromium substrate 12 are bonded with a single application of heat and pressure, the diffusion bond depths between the titanium interlayer 16 and the porous tantalum structure 10 and between the titanium interlayer 16 and the cobalt-chromium substrate 12 may be different.

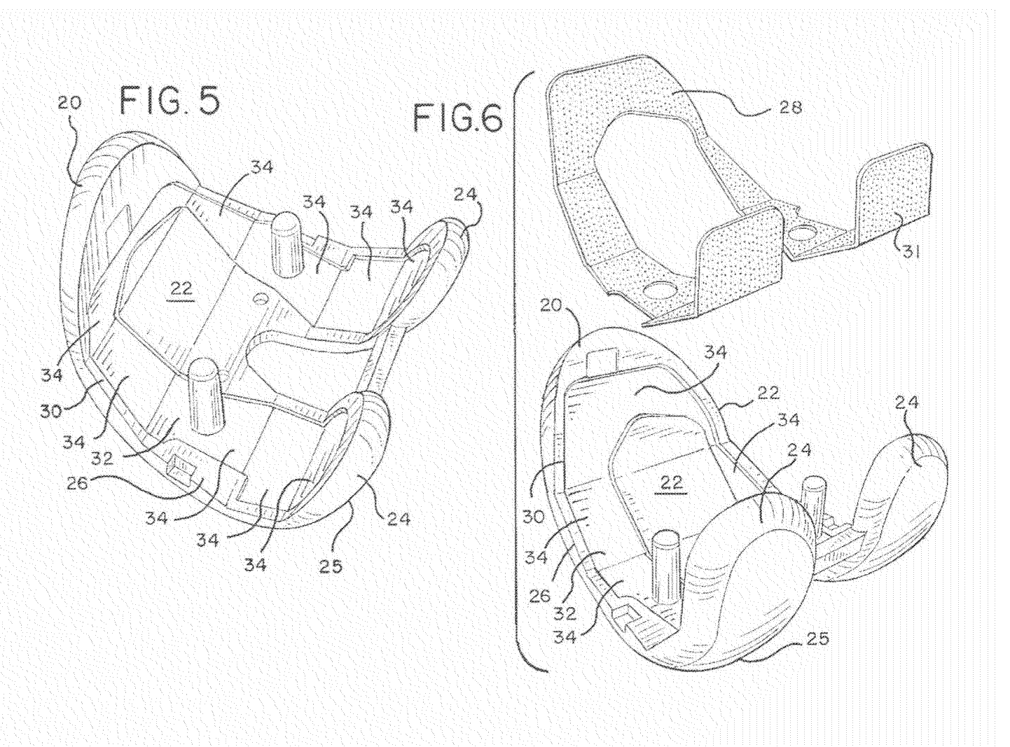

FIGS. 5 and 6 illustrate one exemplary embodiment of a substrate having a geometrically complex surface. In particular, the illustrated substrate is a cobalt-chromium femora knee implant 20. Although the following is described with reference. to femoral implant 20, the substrate having a geometrically complex surface may be any cobalt or cobalt-chromium substrate, such as those used as ankle, shoulder, wrist, finger, toe, hip and elbow implants. Femoral knee implant 20 includes a main body portion 22 and a pair of condyle members 24 extending therefrom. Implant 20 also includes a bottom surface 25 for articulating against a tibial implant and a top surface 26 which is configured to interface with the femur. Generally, a porous tantalum structure 28 (FIG. 6) is bonded by an interlayer (not shown) to top surface 26. Top surface 28 includes a recessed generally U-shaped section 30 that is configured to receive the similarly shaped porous tantalum structure 28. In the illustrated embodiment, U-shaped section 30 includes a geometrically complex surface 32 that has nine flat sections 34 wherein each flat section extends at an angle relative to adjacent flat sections.

In one embodiment of a process of bonding porous tantalum structure 28 to surface 32, the interlayer may be coated, for example by plasma spray, to either surface 32 of implant 20 or surface 31 of porous tantalum structure 28. After the coated interlayer has been applied, any of the diffusion bonding processes described herein may then be used to bond porous tantalum structure 28 and implant 20 to the interlayer.

As discussed above, it may be difficult to coat a uniform interlayer having a consistent thickness to geometrically complex surface 32. In such instances, the interlayer may be coated, for example by plasma spraying, onto surface 31 of porous tantalum structure 28.

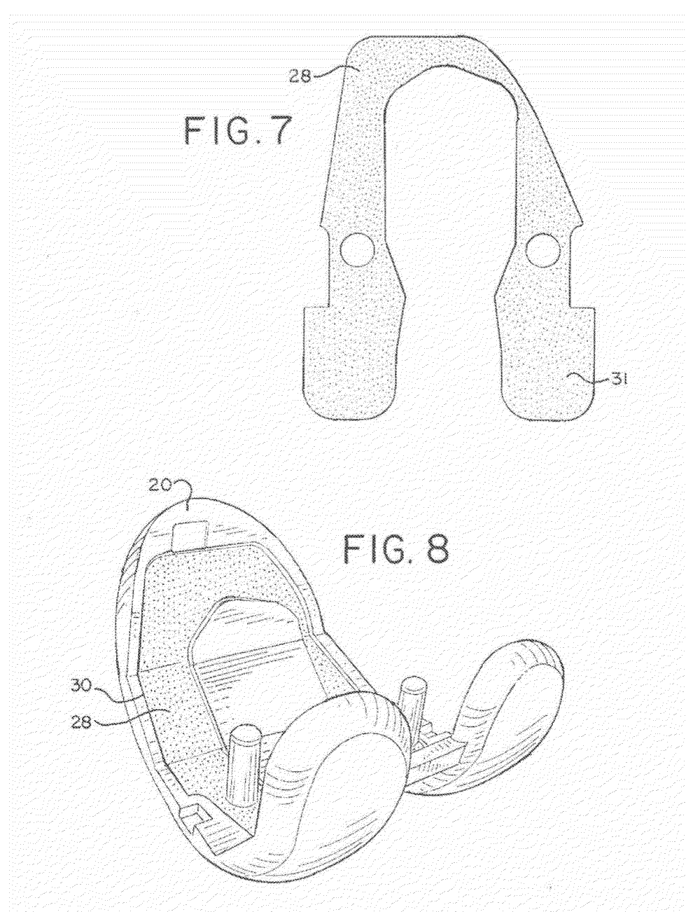

Referring to FIG. 7, porous tantalum structure 28 may have a first or initial configuration, such as the substantially flat configuration shown in this figure. While in this substantially flat configuration, the interlayer (not shown) may be coated onto surface 31 of porous tantalum structure 28 wherein surface 31 will be the surface bonded to surface 32 of implant 20 via the interlayer. The interlayer may be coated onto surface 31 of porous tantalum structure 28 by, for example, plasma spraying. Coating the interlayer onto porous tantalum structure 28 while structure 28 is in the substantially flat configuration makes it easier to achieve an interlayer with a substantially uniform thickness.

After the interlayer has been coated onto surface 31 of porous tantalum structure 28, structure 28 is then bent into a second configuration. In the embodiment illustrated in FIG. 6, porous tantalum structure 28 is bent so that the shape of structure 28 is substantially congruent to recessed section 30 and geometrically complex surface 32 of implant 20. In this particular embodiment, porous tantalum structure 28 is bent so that surface 31 has nine substantially flat sections corresponding to the nine substantially flat sections 34 of surface 32. Porous tantalum structure 28 is placed in recessed U-shaped section 30 so that the interlayer coated on surface 31 of structure 28 is placed in contact with surface 32 of implant 20. Any of the diffusion bonding processes described herein may then be used to bond porous tantalum structure 28 and implant 20 to the interlayer to form the construct illustrated in FIG. 8.

As discussed above, when an interlayer is porous, the porosity may allow the interlayer to be a compressible interlayer. For example, a plasma sprayed interlayer may include a porosity which allows the interlayer to be compressible. When sufficient pressure is placed on the porous interlayer, the pores of the interlayer collapse resulting in compression of the interlayer. In one embodiment, the compressible interlayer is compressed during the diffusion bonding process. In particular, during diffusion bonding, heat and pressure are applied to the substrate, porous tantalum structure and the interlayer to bond the same together. The pressure applied during this bonding process may be sufficient to collapse the pores of the interlayer so as to compress the interlayer. Compression of the interlayer or portions thereof results in the thickness of the interlayer or portion thereof being less than the thickness in the original uncompressed state. The interlayer may be uniformly compressed across the interlayer or may be non-uniformly compressed such that only certain areas or sections of the interlayer are compressed. FIG. 10 is a photomicrograph illustrating one embodiment of a construct shown after the diffusing bonding process. The construct includes a porous tantalum construct 12′, a compressed interlayer 16′ and a substrate 10′. As shown in this figure, the pores of compressed interlayer 16′ are collapsed.

Such a compressible interlayer may advantageously assist in providing a substantially complete bond between the substrate and tantalum porous structure across substantially all of the facing surfaces of the substrate and tantalum structure. In some applications, such as when the porous tantalum structure is bonded to a geometrically complex surface of a substrate, there may be deviations from the geometrical congruencies between the substrate and the porous tantalum structure. Such deviations may include deviations from parallelism, unintended curvature, and dimensional mismatch. When such deviations exist and the interlayer is substantially incompressible, for example when the interlayer is a substantially solid sheet, bonding quality between the tantalum porous structure and the substrate may be poor and unequal across the surfaces and the tantalum porous structure may not completely bond to the substrate. On the other hand, when such deviations exist and the interlayer is a compressible interlayer, the compression of the interlayer compensates for such deviations, resulting in a relatively higher quality bond in which the bond between the porous tantalum structure and the substrate is substantially complete.

### EXAMPLE

A comparison was made to determine if there were any differences in the bonding between constructs formed by bonding porous tantalum structures to substrates with compressible interlayers and with incompressible interlayers. The porous tantalum structures used in this comparison are available from Zimmer, Inc., Warsaw, Ind., and sold under the trademark Trabecular Metal®. Additionally, the cobalt-chromium femoral knee implants used this comparison are similar to those shown in FIGS. 5 and 6 and are also available from Zimmer, Inc., Warsaw, Ind.

A solid, nonporous substantially incompressible interlayer sheet of titanium having a thickness of about 0.020 inches (0.51 mm) was employed in a diffusion bonding process to bond a porous tantalum structure having a thickness of about 0.045 (1.1 mm) and a porosity of about 80% to the geometrically complex surface of a femoral implant. The bonding process included placing the sheet interlayer between the porous tantalum structure and the substrate and simultaneous bending of the sheet interlayer to the substrate, and the porous tantalum to the sheet interlayer. The diffusion bonding process included about 1000 lbs of fixture pressure using a multi-piece compression tool, and bonding at 940° C. (1725° F.) for approximately one hour in a vacuum environment.

A porous compressible layer was used in a diffusion bonding process to bond a second porous tantalum structure having a thickness of 0.045 inches (1.1 mm) and a porosity of 80% to the geometrically complex surface at a second femoral implant. The bond no process included using a plasma sprayer available from Orchid Bio-Coat, Southfield, Mich. to plasma spray a titanium porous compressible interlayer onto the a surface of the second porous tantalum structure while the second porous tantalum structure was provided substantially flat configuration, such as the configuration shown FIG. 7. The plasma sprayed interlayer had a thickness of approximately 0.02 inches and a porosity of approximately 30% to 40%. The substantially flat porous tantalum structure was then bent so that the coated surface of the tantalum structure substantially corresponded with the geometrically complex surface of the femoral implant. The interlayer on the coated surface of the porous tantalum structure was then placed in contact with the geometrically complex surface of the femoral implant and bonded thereto by diffusion bonding to form a second construct. The diffusion bonding process included about 1000 lbs of fixture pressure using a multi-piece compression tool, and bonding at 940° C., (1725° F.) for approximately one hour in a vacuum environment.

The bonding quality of each construct was then assessed by laser holography as described in for example U.S. Pat. No. 4,408,881, which is hereby incorporated by reference. FIG. 12 shows the laser holography image for the first construct including the incompressible interlayer and FIG. 13 shows the laser holography image from the second construct including the compressible interlayer. The light grey areas indicate a quality bond between the porous tantalum structure and the implant, and the dark black areas indicate that no bond has formed between the porous tantalum structure and the implant in that particular area. As can be seen from these figures. FIG. 12 includes large areas of nonbonding and FIG. 13 includes few if any areas of nonbonding.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method and illustrative examples shown and described. Accordingly, departures may be made from such details without departing from the scope of the general inventive concept.

1. (canceled)

2. A method of bonding, comprising:
providing a substrate comprising cobalt or cobalt-chromium;
providing a subassembly that includes a porous tantalum structure with a compressible interlayer formed on a surface portion of the porous tantalum structure, said compressible interlayer having a porosity of between 5% and 40% and consisting essentially of interconnected metal or metal alloy particles that exhibit solid solubility with tantalum and with cobalt or cobalt-chromium, said interconnected metal or metal alloy particles defining collapsible pores therebetween;
bending the subassembly from a first configuration to a second configuration;
forming an assembly which includes placing an exposed surface of the compressible interlayer in contact with the substrate after said bending; and
applying heat and pressure to the assembly for a time sufficient to achieve solid-state diffusion between the substrate and the compressible interlayer and between the compressible interlayer and the porous tantalum structure.

3. The method of claim 2, wherein the compressible interlayer has a substantially uniform thickness before said bending.

4. The method of claim 2, wherein said applying heat and pressure to the assembly includes compressing a portion of the compressible interlayer from a first thickness to a second, reduced thickness.

5. The method of claim 2, wherein said applying heat and pressure to the assembly includes collapsing one or more of said collapsible pores.

6. The method claim 2, wherein said compressible interlayer is a coating layer formed by plasma spraying.

7. The method of claim 2, wherein the compressible interlayer is formed by plasma spraying in at least a partial vacuum so that the compressible interlayer has a porosity of between 20% and 40% before said applying heat and pressure to the assembly.

8. A method of bonding, comprising:
providing a substrate comprising cobalt or cobalt-chromium;
providing a subassembly that includes a porous tantalum structure with a compressible interlayer formed on a surface portion of the porous tantalum structure, said compressible interlayer consisting essentially of a metal or a metal alloy that exhibits solid solubility with tantalum and with cobalt or cobalt-chromium;
bending the subassembly from a first configuration to a second configuration;
forming an assembly which includes placing an exposed surface of the compressible interlayer in contact with the substrate after said bending; and
applying heat and pressure to the assembly for a time sufficient to achieve solid-state diffusion between the substrate and the compressible interlayer and between the compressible interlayer and the porous tantalum structure.

9. The method of claim 8, wherein the compressible interlayer has a substantially uniform thickness before said bending.

10. The method of claim 8, wherein said applying heat and pressure to the assembly compresses the compressible interlayer.

11. The method of claim 10, wherein the compressible interlayer is uniformly compressed.

12. The method of claim 8, wherein the compressible interlayer is formed on the surface portion of the porous tantalum structure without significantly occluding one or more pores of the porous tantalum structure.

13. The method of claim 8, wherein the porous tantalum structure is substantially flat in the first configuration of said subassembly.

14. The method of claim 8, wherein the porous tantalum structure in the second configuration of said subassembly is substantially congruent with a non-planar surface of the substrate.

15. The method of claim 14, wherein said non-planar surface includes two or more flat sections that project at an angle with respect to each other.

16. The method of claim 14, wherein said non-planar surface includes a rounded surface.

17. The method of claim 8, wherein the compressible interlayer has a porosity of at least 5% prior to said applying heat and pressure to the assembly.

18. The method of claim 8, wherein the compressible interlayer has a porosity of at least 20% prior to said applying heat and pressure to the assembly.

19. The method of claim 8, wherein the compressible interlayer has a thickness of at least 0.010 inches prior to said applying heat and pressure to the assembly.

20. The method of claim 8, wherein the compressible interlayer has a thickness of at least 0.020 inches prior to said applying heat and pressure to the assembly.

21. The method of claim 8, wherein the compressible interlayer consists essentially of a metal or a metal alloy selected from hafnium, manganese, niobium, palladium, zirconium, titanium, or alloys or combinations thereof.

22. The method of claim 8, wherein the compressible interlayer is formed by thermal spraying, plasma spraying, electron beam deposition, laser deposition, cold spray, chemical vapor deposition, or electrode position.

23. The method of claim 8, wherein the compressible interlayer is formed onto the surface portion of the porous tantalum structure by a process that includes applying liquid particles of said metal or said metal alloy to the porous tantalum structure and solidifying said liquid particles.

24. The method of claim 8, wherein the substrate forms part of an ankle, elbow, shoulder, knee, hip, wrist, finger, or toe implant.
